(19) United States
(12) Patent Application Publication
(10) Pub. No.: US 2012/0330234 A1
(43) Pub. Date: Dec. 27, 2012

(54) DEVICE FOR HEATING FLOWING FLUIDS AND PRODUCTION METHOD

(75) Inventors: Michael Balluff, Bielefeld (DE); Holger Teschner, Bielefeld (DE)

(73) Assignee: BARKEY GMBH & CO. KG, Leopoldshoehe (DE)

(21) Appl. No.: 13/581,852

(22) PCT Filed: Mar. 16, 2011
(86) PCT No.: PCT/DE2011/000261

§ 371 Date: Sep. 12, 2012

(30) Foreign Application Priority Data
Mar. 16, 2010 (DE) 10 2010 002 895.9
May 25, 2010 (DE) 10 2010 029 274.5
Jun. 7, 2010 (DE) 10 2010 029 732.1
Jul. 7, 2010 (DE) 10 2010 031 068.9

(51) Int. Cl. A61M 5/44; B05D 5/12; B05D 3/06; B23P 17/04

(52) U.S. Cl. 604/114; 29/527.2; 427/58; 427/555

(57) ABSTRACT

The invention relates to a device for heating flowing fluids, in particular intravenous fluids, comprising a fluid housing containing at least one fluid channel, through which the fluid can be conducted from an inlet of the fluid housing to an outlet of the fluid housing, a heating unit containing at least one electric flat heating element for heating the fluid flowing through the fluid channel, and a temperature control unit containing at least one temperature sensor arranged on the flat heating element, wherein the flat heating element is arranged inside the fluid housing, wherein the flat heating element at least partially forms a wall of the fluid channel, wherein at least one linear fluid channel extends between the inlet of the fluid housing and the outlet of the fluid housing.

This present invention relates to a device for heating flowing fluids, in particular intravenous fluids, comprising a fluid housing containing at least one fluid channel through which the fluid can be conducted from an inlet of the fluid housing to an outlet of the fluid housing, a heating unit comprising at least one electric flat heating element for heating fluid that flows through the fluid channel and a temperature control unit containing at least one temperature sensor arranged on the flat heating element, wherein the flat heating element is arranged inside the fluid housing and at least partially forms a wall of the fluid channel.

The invention also relates to a process for manufacturing a flat heating element to be used for heating flowing fluids.

Prior known from DE 198 28 923 B4 is a device for heating flowing fluids which comprises a fluid housing having a fluid channel formed therein for conducting fluid from an inlet of the fluid housing to an outlet thereof. For heating the fluid passed through the fluid housing there is a heating unit provided that contacts on outer surface of the fluid channel and/or the fluid housing by an electric flat heating element. Said flat heating element is an electric resistance heating unit comprising a rigid printed board on one flat side of which a metallic heat conductor track extends. This heat conductor track is meandering such that an improved heat transfer is ensured due to an overlap of heat conductor track and fluid channel.

WO 2005/027578 A1 is disclosing a device for heating flowing fluids comprising a fluid housing with a fluid channel having a serpentine (meander type) or helical configuration which extends from a face end inlet of the fluid housing to an opposing face end outlet thereof. Heat conductor plates are provided as heating units which are arranged on opposing flat sides of the fluid housing. Electric flat heating elements are disposed within said heating conductor plates which comprise a rigid printed board to which heat conductor tracks have been formed by lithographic processing. Temperature sensors also can be arranged on the printed board to measure fluid temperature such that in coaction with a temperature control unit the temperature of the fluid may be controlled to a predetermined temperature level.

It is an object of this present invention to improve a device for heating flowing fluids in such a way that heat input into the fluid housing is improved and homogeneous and efficient heating will be ensured in a simply way.

To achieve this object this present invention is in conjunction with the preamble of Patent claim 1 characterized in that the flat heating element comprises a flexible or rigid printed board with heat conductor tracks arranged on the first flat side (118′) and/or second flat side (118″) wherein the flat heating element is arranged inside the fluid housing and at least partially forms a wall of the fluid channel.

The invention affords the advantage that the fluid channel is just defined by the configuration of the fluid housing with a flat side of the flat heating element forming one wall of the fluid channel and that a fluid channel can be easily provided this way which permits the flat heating element to act direct on the fluid passing the fluid channel.

According to the invention the inner surface of the fluid housing is profiled such as to form a meander or spiral type fluid channel. The flat heating element may be even. This means that the input heat as transferred may be higher and/or the heating device may be of greater compactness.

The flexible heating element has a printed board on an at least one flat side of which a metallic heat conductor track (resistance heating track) is provided for heat generation. Said printed board may be disposed on an inner surface of a fluid housing wall and/or between two opposing inner surfaces of the fluid housing in a spaced relation from said housing wall. The printed board can hence be part of a configuration of the fluid channel such that different fluid channel structures may be readily obtained. In particular, a heat transfer factor referred to a longitudinal extension of the fluid housing may be increased.

According to another modification of the invention the flat heating element is integral with a contact strip which extends along a longitudinal side of the fluid housing on the outside thereof. The housing halves are preferably bonded to each other with the flat heating element in between, for instance with an adhesive sealant used in the area of the contact strips. This permits to provide a hermetically closed fluid housing.

Another modification of this present invention provides for a cassette consisting of fluid housing and flat heating element to be inserted into a slot of a control unit. The contact rail is snap connected to corresponding contact elements of the control unit while in inserted position. The fluid housing is arranged substantially outside said control unit to make it visible from outside. The advantage this offers is that flow disturbances such as the formation of gas bubbles may be detected. To dispel gas bubbles the slot of the control unit is preferably vertical such as to have the cassette attain an upright position while inserted and to make the fluid pass through the cassette in vertical direction from bottom to top.

According to another modification of this invention the printed board of the flat heating element is a flexible plastic film to which the heat conductor tracks are formed by laminating and then structured by photolithographic or laser methods. The plastic film may be a temperature resistant polyimide sheet having a thickness in the range from 25 μm and 125 μm. The heat conductor tracks are preferably made of a biocompatible conductive material such as one from the groups of aluminum, aluminum alloys, gold or alloy steel to ensure biocompatibility. Alternatively, the heat conducting tracks may consist of a copper or any other high-resistivity material that is preferably provided with a biocompatible insulating layer.

A further modification of this present invention provides for the inlet and the outlet to be disposed on a first side of the fluid housing while a contact strip fixed to the flat heating element is arranged on a second side thereof. Said contact strip extends outside the fluid housing and enables the flat heating element to be plugged to a connector block of a control and/or regulating unit. The fluid housing can for instance be inserted into a slot of the regulating unit and both mechanically and electrically connected thereto via said contact strip. This means that the heating unit may be easily brought into an operative position and/or removed therefrom.

This is important particularly where the heating unit is a throw-away article having a restricted service life.

According to a still further modification of the invention is the flat heating element on one flat side provided with at least two heat conductor sections which are assigned to at least two different contacts and which are tandem arranged in main flow direction. This affords the advantage that different regions of the fluid channels may be differently heated in a selective move to permit a defined heating control as from case to case required.

In a still further modification of this invention a quotient of the effective heat transfer surface of the flat heating element divided by a flat element surface area projected in a longitudinal axis of the fluid housing is larger than 1. The advantage here is that a heat transfer referred to the area of the fluid housing can be essentially increased. The flat heating element and/or the fluid channel may for instance extend from the inlet to the outlet of the fluid housing in corrugated configuration in which case substantially the full width of the fluid housing is preferably being used. Such a wavy configuration of the flat heating element with a relatively small bending radius permits the device to be in the form of a compact unit which has a relatively high efficiency referred to the area of the fluid housing.

Another modification of the invention provides for the flat heating element to comprise flat heating segments (heat conductor sections) that can be optionally cut in or out such that dependent on a specific application in each case a higher or lower temperature of the fluid may be set.

According to a still further modification of this invention there are a number of temperature sensors arranged on the printed board. A sensor unit to assess a flow velocity of the fluid may be provided on said printed board to thereby improve the regulating result even further. A regulating means adapted to set a fluid heat rate is preferably arranged in a separate regulating unit and electrically connected thereto via contacts of the flat heating element.

The printed board preferably has a relatively low thermal capacity which improves the temperature regulating properties. It is due to a homogeneous temperature distribution inside the flat heating element that an increased heat input is obtained at a reduced working temperature of the flat heating element.

A preferred embodiment of the flat heating element provides for the heat conductor tracks on different flat sides of said heating element to be arranged in an offset relation to each other such that a uniform heat is generated via the area of the flat heating element and/or the plastic film, but radiated to both sides. This affords the advantage that an improved area utilization of the plastic film with heat conductor tracks can be achieved.

Still another modification of the flat heating element provides for several heat conductor tracks to be arranged in fluid flow direction which can be separately activated such that a plurality of heat conductor sections are formed along the fluid channel. This permits the input of heat to be accomplished by alternating and/or periodical activation of different heat conductor tracks. An advantage here is that a defined heat input can be achieved at lower momentary current loads on the heat conductor tracks.

To carry the process of the invention into effect it is in conjunction with the preamble of Patent claim 12 characterized by the following steps: Applying an electrically conductive layer on one and/or two flat sides of a substrate to provide a semi-product that is made available from a roll; sequential structuring of heat conductor tracks on semi-product sections by lasing or photolithographic methods while a length of semi-product is being unwound from one roll and wound up on another; sequential fitting of semi-product lengths with a number of sensor elements; cutting the semi-product sections to a size of specific flat heating elements; coating the flat heating elements with a biocompatible material and/or an electrically insulating material and/or a mechanical protective material and/or a thermally conductive layer.

The particular advantage of the process according to this present invention resides in that the processing steps proposed are applicable to semi-finished materials made available from a roll. Applying an electrically conductive layer to a substrate can be performed in a roll-to-roll process wherein both the flexible substrate and the electrically conductive layer material are unwound and joined for instance by laminating and/or bonding and thereafter wound up again as a semi-finished product. It is while coiling and uncoiling that heat conductor tracks can be sequentially formed and/or a number of sensors be fitted to the semi-finished stock. Finally, the size of a specific flat heating element can be obtained by cutting appropriate blanks of semi-finished material from the material web. Covering and/or coating the flat heating element with a biocompatible material and/or an electrically insulating material and/or a mechanical protective material and/or a thermally conductive layer can be done prior to cutting blanks from the web or after such severing to provide then separated flat heating elements. It is an advantage that the process of the invention permits to produce flat heating elements at low cost and in a dependable way.

Still another modification of the process according to this present invention provides for heat conductor tracks to be formed on both sides of the semi-finished material such that as viewed in projection to the flexible substrate they are overlapping each other or disposed side by side. Offset structuring may be easily achieved on both flat sides simultaneously during the process of unwinding the semi-finished material with the roll movement stopped while this processing step is being performed.

Further advantages of this present invention are as disclosed in the further subclaims.

Exemplary embodiments will now be described in closer detail with reference to the drawings.

In the drawings:

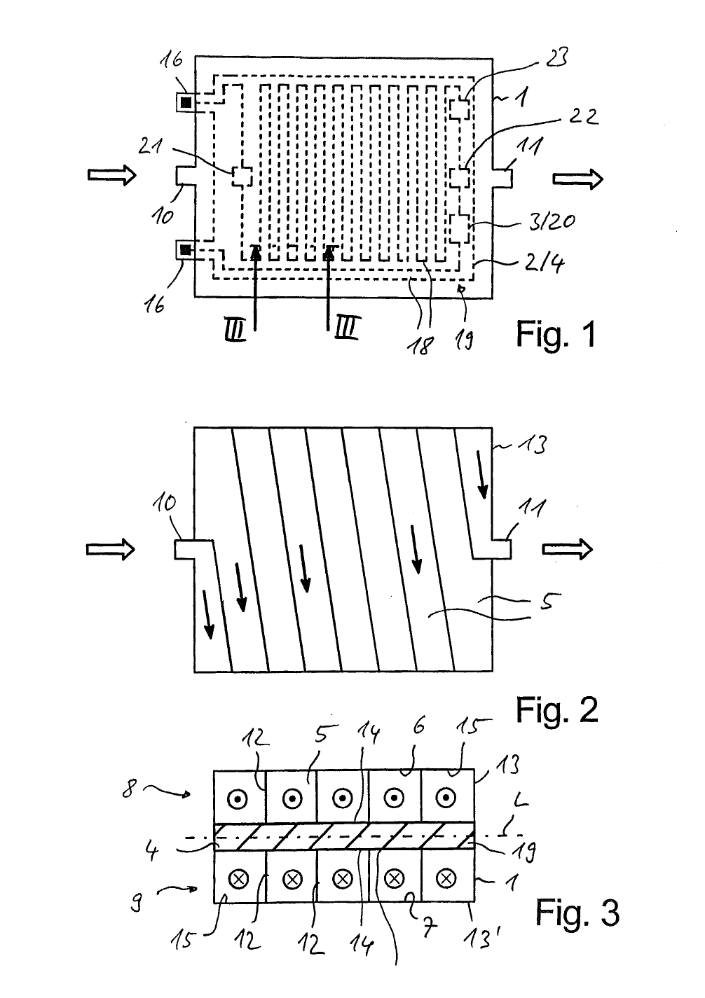

FIG. 1 is a schematic top view of a device according to the present invention in a first embodiment comprising a fluid housing in which an electric flat heating element shown in dashline representation is arranged;

FIG. 2 is an inside view of half a fluid housing with guide walls projecting from an inner surface thereof to form a fluid channel;

FIG. 3 is a section along line III-III in FIG. 1;

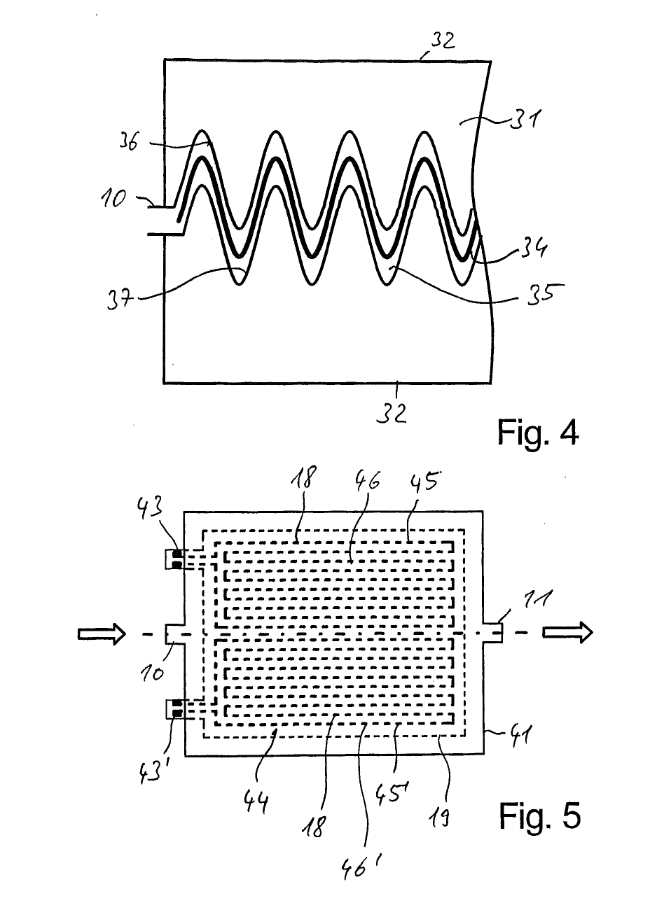

FIG. 4 is a longitudinal section as per FIG. 3 through a device in a second embodiment;

FIG. 5 is a top view of a device in a third embodiment which shows an electric flat heating element in a segmented representation;

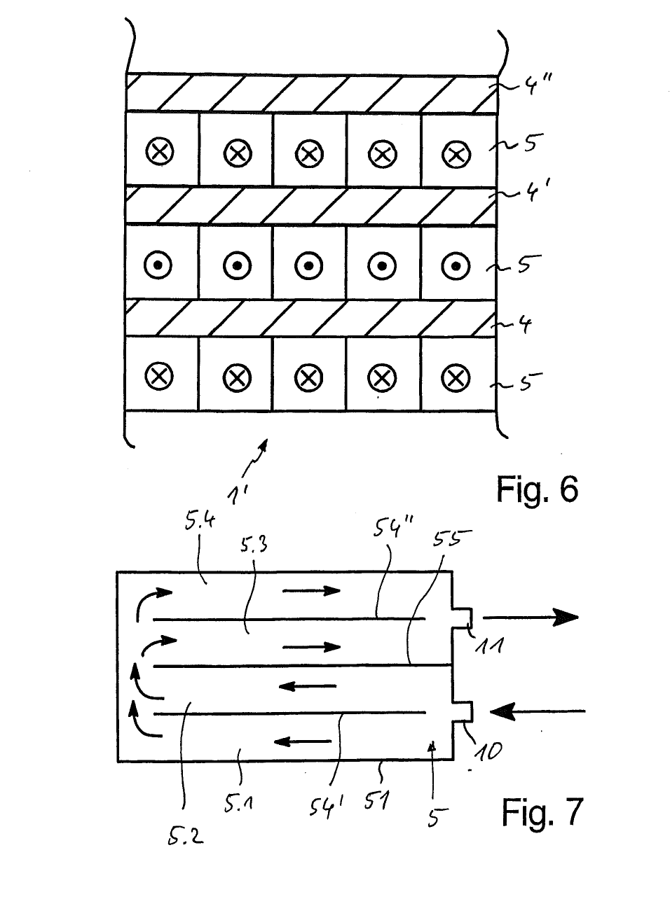

FIG. 6 is a partial section through a fluid housing with a plurality of flat heating elements in sandwich arrangement;

FIG. 7 is a longitudinal section through a device of this present invention in another embodiment;

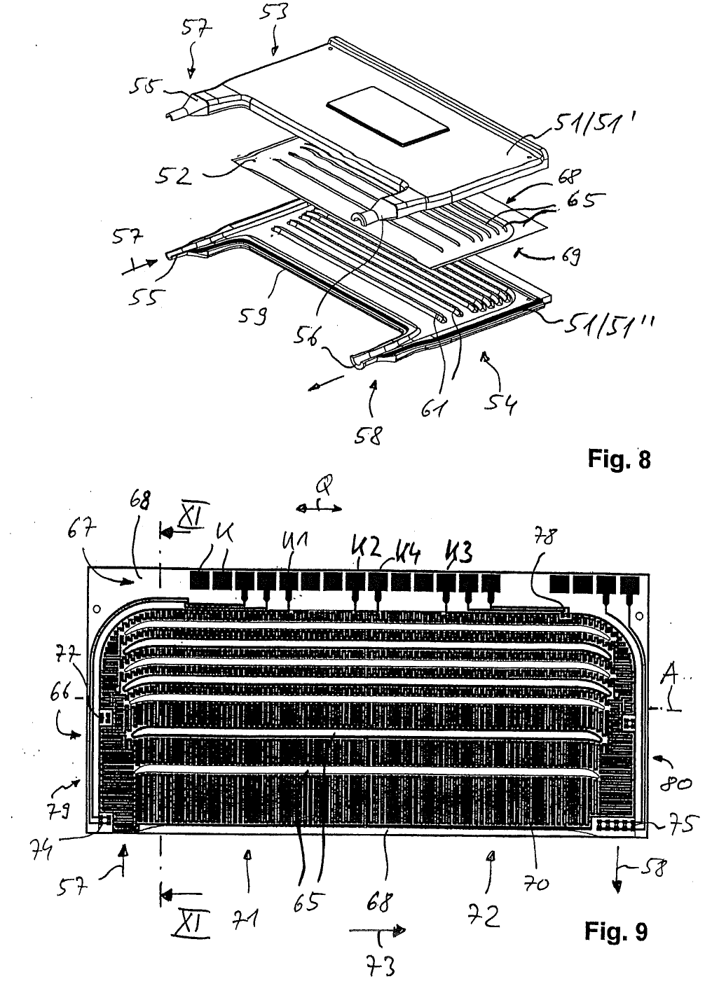

FIG. 8 is an exploded view of a fluid housing in a still further embodiment;

FIG. 9 is a top view of a flat heating element inserted into a fluid housing as per FIG. 3;

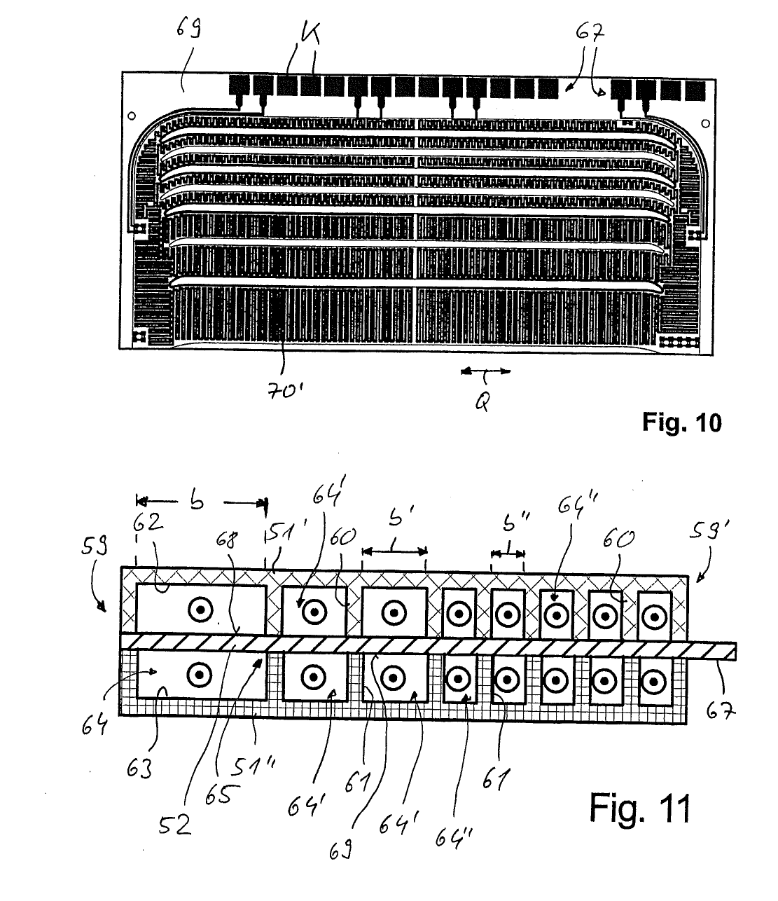

FIG. 10 is a bottom view of the flat heating element shown in FIG. 9;

FIG. 11 is a schematic cross-section through a fluid housing containing the flat heating element shown in section along line XI-XI in FIG. 9;

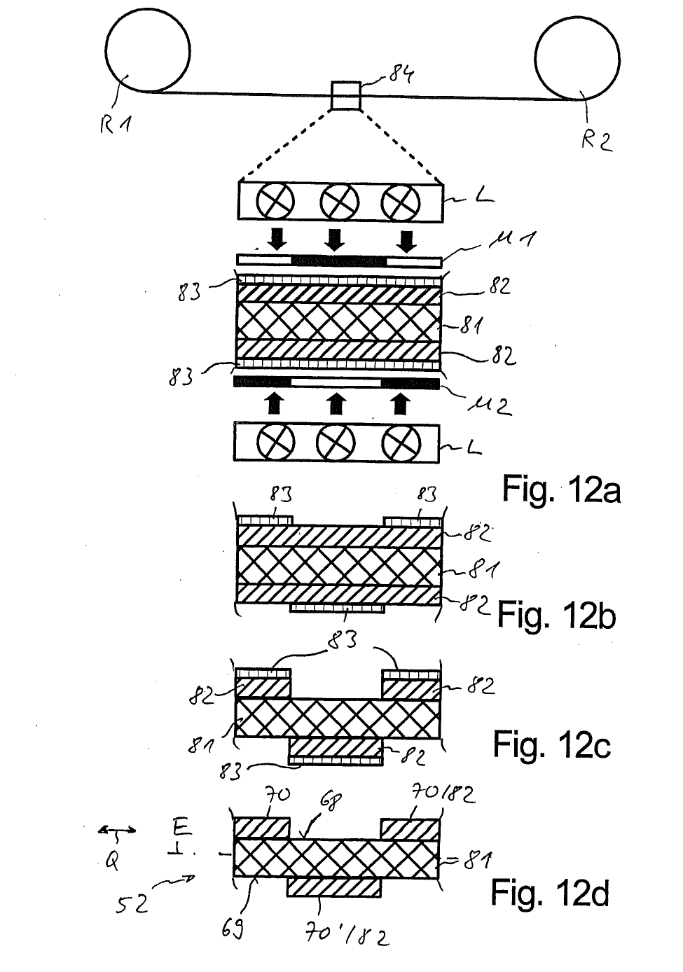

FIG. 12a is a schematic representation of a semi-finished material made available on a roll which is provided with a photosensitive layer and a masking in a first processing step;

FIG. 12b is a cross-section through a detail of the semi-finished material shown FIG. 12a after development of the photosensitive layer;

FIG. 12c is a cross-section through the semi-finished material shown in FIG. 12b after an etching process;

FIG. 12d is a schematic cross-section through the semi-finished material shown in FIG. 12c after removal of the photosensitive layer from the now exposed heat conductor tracks;

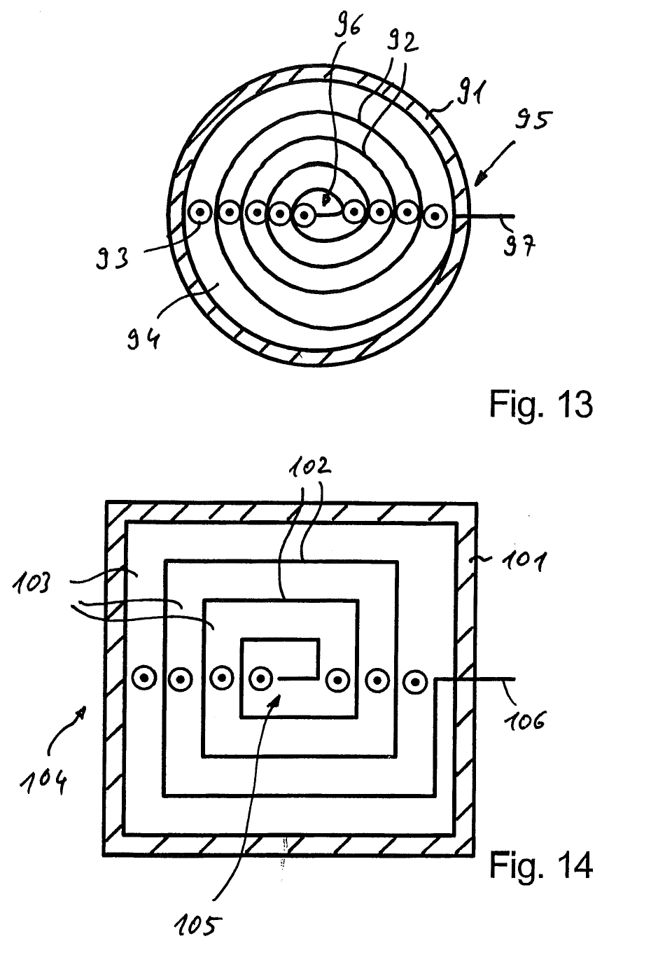

FIG. 13 is a schematic cross-section through a cylindrical fluid housing with a helical flat heating element;

FIG. 14 is a schematic cross-section through a fluid housing of rectangular section with a helical flat heating element;

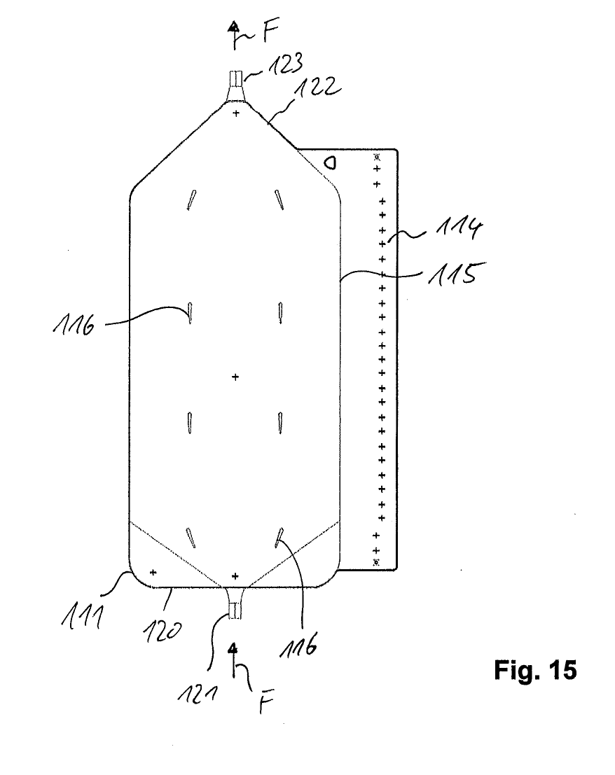

FIG. 15 is a schematic top view of a device according to the invention in a further embodiment comprising a fluid housing and a contact section of a flat heating element that protrudes from a longitudinal side;

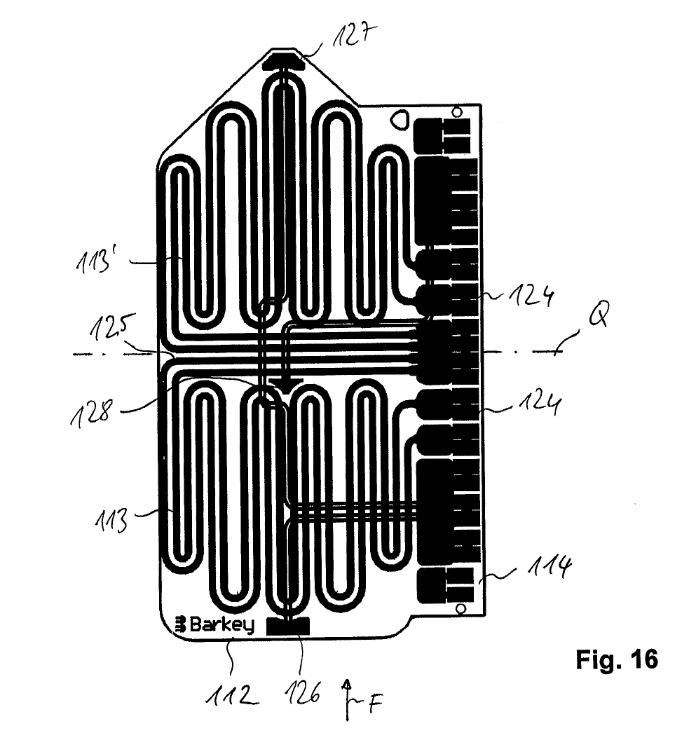

FIG. 16 is a top view of the flat heating element inserted as per FIG. 15;

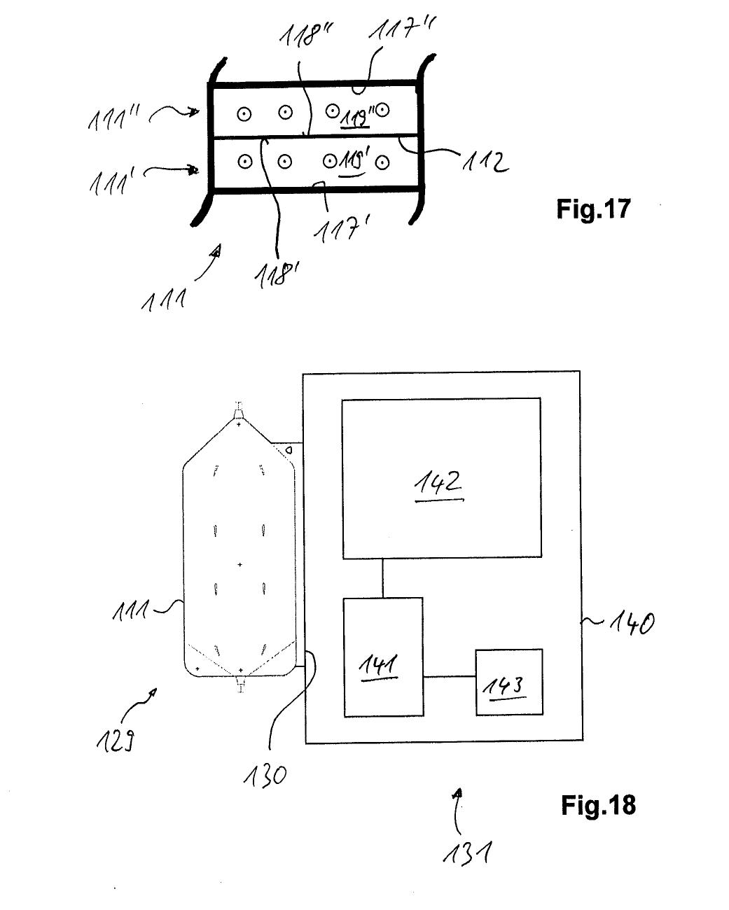

FIG. 17 is a partial cross-section through the fluid housing; and

FIG. 18 is a schematic representation of a device according to the invention as per FIG. 15 in a plugged state in which the contact section of the flat heating element is inserted into a receiving slot of a control unit.

A device of this present invention for heating flowing fluids is preferably used for intravenous fluids (type IV fluids). This device of the present invention for instance serves to heat flowing blood or an infusion fluid. The device permits to heat a fluid while it is passing from a fluid container to a patient.

In a first embodiment of the invention the device substantially comprises a fluid housing 1, a flexible electric heating unit 2 and a temperature control unit 3.

The heating unit 2 has a flexible electric flat heating element 4 for heating a fluid passing through a fluid channel 5 of the device which element is disposed inside the fluid housing 1. The flexible electric flat heating element 4 operating in the resistance mode is substantially even and disposed centrally between a first inner surface 6 and a second inner surface 7 of the fluid housing 1. Said flat heating element 4 separates the relative flat and rectangular fluid housing 1 into two halves with a first fluid channel section 8 extending between said first inner surface 6 and the flat heating element 4 and a second fluid channel section 9 extending between said second inner surface 7 and the flat heating element 4 through which fluid passes in alternating directions as viewed in flow direction—see directional signs in FIG. 3. This means that a helical fluid channel 5 is formed between an inlet 10 in one face end of the fluid housing 1 and an outlet 11 in the other face end of said housing 1. Since the flat heating element 4 is preferably disposed centrally between said first inner surface 6 and said second inner surface 7 said first fluid channel sections 8 and said second fluid channel sections 9 extending along either side of a longitudinal center plane L of the fluid housing 1 are of identical cross-sections.

The flat heating element 4 contacts each of the marginal edges of guide walls 12 of one of two tub-shaped halves 13, 13′ of the fluid housing and is thus stably arranged between said halves 13, 13′ of the fluid housing 1. The fluid channel 5 is hence of substantially rectangular cross-section wherein one surface of the flat heating element 4 forms a first side wall 14 while the inner surfaces 6 and/or 7 of the housing halves 13, 13′ provide an opposing second side wall 15 and wherein said side walls are joined by guide walls 12 which are integral with said housing halves 13, 13′ and in the form of a third side wall. Said housing halves 13, 13′ are hence profiled on their insides.

The flat heating element 4 has two contact studs 16 protruding outwardly from the face end of the fluid housing 1 such that the flat heating element 4 can be brought into contact with an electric voltage source.

The flat heating element 4 has a flexible printed board 19 to the two flat sides 17 of which a metallic heat conductor track 18 is applied. Said flexible printed board 19 is preferably a film of a plastic material, for instance a polyimide, which can have a thickness between 25 μm and 100 μm. The heat conductor tracks 18 may be formed on the plastic film in a laminating or photolithographic process either by etching or screen printing. In particular, a metal laminated plastic film may be provided on which the heat conductor tracks are formed by photolithographic methods. The plastic film may consist of an elastomeric (silicone gum) or thermoplastic material. The plastic film serving as substrate may for instance consist of a polyimide (Kapton), a polyester or preferably a transparent mylar. In a first alternative of the invention can the heat conductor track consist of an aluminum material and/or aluminum alloy, gold or alloyed steel such that a direct contact is made possible with the fluid in a biocompatible way. Another alternative provides for the heat conductor track to consist of a copper material which is initially provided with a further biocompatible protective layer to suppress the toxicological effect of the copper. Also can the heat conductor tracks be formed of a high-resistivity material and/or resistance alloy such as a copper alloy or a copper-nickel alloy (constantan).

According to an alternative of the invention can the temperature control unit 3 be arranged on the flexible printed board 19. It comprises a control unit 20 with a microprocessor and/or micro-controller and a plurality of temperature sensors of which a first sensor 21 is arranged in the area of the inlet 10, a second sensor 22 in the area of the outlet and a third temperature sensor 23 to assess excess temperature conditions in any place of the printed board 19. It is possible also to provide just two temperature sensors 21, 22. Moreover, the printed board 19 may be provided with a not-shown sensor to assess a fluid flow rate. Heating temperature control is substantially achieved inside the fluid housing 1 with the contact studs 16 only to establish an electric connection to an external voltage source. Repeat use of the device according to this present invention is ensured by having the fluid housing 1 and/or the flat heating element 4 sterilized.

In a not-shown alternative embodiment can the fluid housing 1 also be a cassette for one-time use in which case the temperature sensors and/or the flow rate sensor only will be arranged on the printed board. Alternatively, the float rate may be assessed by evaluating measured values made available by the temperature sensors also. This provides an information at least on whether fluid is flowing or not. The control unit for temperature control is disposed outside the fluid housing 1 wherein electric connection to the sensors arranged on the flexible printed board is established via appropriate contact studs 16.

Other than in the first embodiment hereinbefore described a further embodiment of the invention according to FIG. 4 provides for not only a fluid housing 31 to be profiled, but also a flat heating element 34 integrated thereinto. Inner surfaces 36, 37 of the fluid housing 31 and the flat heating element 34 are corrugated such that following entry into the fluid housing 31 the fluid is at the inlet 10 fed toward the opposing outlet 11 in an alternating mode between two opposing outer flat side 32 of the fluid housing 31. The wavy fluid channel 35 thus formed has a length that is a multiple greater than the length of the flat side 32 of the fluid housing 31 that extends between the inlet 10 and the outlet 11. This affords the advantage that heat transfer from the flat heating element 34 to the fluid is improved. Said flat heating element is relatively thin to thereby make it substantially compact. The corrugated flat heating element 34 has a comparatively small bending radius at its tips. The relation between the overall area of the flat heating element 34 and the area thereof as projected to the longitudinal center plane L of the fluid housing 1 is larger than 1. A proportionate area of the flat heating element 34 that extends cross to the fluid housing 31 and/or to the flat heating element 34 is hence larger than a proportionate area that extends along said fluid housing 31 and/or said flat heating element 34.

Identical components and/or component functions of exemplary embodiments herein described are denoted by identical reference signs.

In another not-shown embodiment it is possible also to have only the flat heating element profiled while the inner surfaces of the fluid housing remain substantially even.

An alternative embodiment of the invention provides for the flat hating element and/or the inner surfaces of the fluid housing to be in a zigzag arrangement. The essential factor here is that the fluid undergoes a directional change cross to and/or toward the longitudinal center plane L.

In a further embodiment of the invention according to FIG. 5 can a flat heating element 44 comprise a number of heating element segments 45, 45′ that can be selectively activated or deactivated such that dependent on a specific application as involved and/or on a specific heat input as from case to case demanded just part or all of the heat conductor tracks 18 are supplied with power. For instance can a first flat heating element 45 comprise a first heat conductor track 46 which extends on a first side of the printed board 19 relative to a transversal center plane Q of the fluid housing 41 and to which electric power can be supplied via a first contact stud 42. On an opposite second side relative to said transversal center plane Q there is a second flat heating element 45′ arranged with a second heat conductor track 46′ to which electric power is supplied via a second contact stud 43. Since just like in the first embodiment according to FIGS. 1 to 3 the fluid channel extends helically around the flat heating element 44 it is possible to ensure uniform fluid heating with just one flat heating element 45, 45′ even.

The fluid housing is preferably transparent.

According to a further embodiment of the invention according to FIG. 6 a fluid housing 1′ may be of sandwich design with a layer of flat heating elements 4, 4′, 4″ and fluid channels 5 is disposed cross to a longitudinal extension of the fluid housing. Fluid channel layers 5 and flat heating element layers 4′, 4″, 4′″ are arranged in alternating order in this sandwich configuration. Said heating element layers 4′, 4″, 4′″ may be fitted with temperature sensors where so deemed necessary. Other than in the embodiment according to FIGS. 1 to 3, therefore, several layers 4′, 4″, 4′″ of flat heating elements are provided between which said layers of fluid channels 5 extend. The fluid channels 5 can be oriented in a longitudinal and/or transversal relation to the inlet 10 and/or outlet 11 of the fluid housing 1.

Other than described for the embodiment according to FIG. 6 it is possible in a further not-shown embodiment to dispose also two fluid channel layers between two layers of flat heating elements. A first fluid channel layer is arranged directly adjacent to a first layer of flat heating elements for the fluid to flow in a first direction. Adjacent to said first fluid channel layer there is a second layer of fluid channels arranged through which the fluid flows in an opposite second direction. This results in a cross-sectional configuration of the fluid housing that conforms to the embodiment according to FIG. 6 wherein the layer of flat heating elements 4′ is omitted such that the layers of fluid channels 5 through which the fluid passes in opposite directions and which are disposed between the fluid channel layers 4 and 4″ are arranged in a direct juxtaposed relation.

Where the flat heating elements 4, 4′, 4″ are provided with heat conductor tracks on only one side it is possible due to a flexible arrangement and/or a good heat conductance that heat input may be effected via the very flat side of the flat heating elements 4, 4′, 4″ that is opposite the flat side on which the heat conductor tracks are arranged.

The embodiment of this invention according to FIG. 7 provides for the inlet 10 and the outlet 11 to be disposed on one common face end of a fluid housing 51. The fluid channel 5 can be split up into two partial fluid channels 5.1, 5.2 which are separated by a flat heating element 54′. Fluid flow is reversed at a face end opposing the inlet/outlet face end via at least two partial fluid channels 5.3, 5.4 which are separated from each other by another flat heating element 54″. The fluid partial streams are brought together in the area of the outlet 11 and discharged via said outlet 11. A partition 55 is provided between the partial fluid channels 5.1, 5.2 on one side that carry fluid in one direction and the partial fluid channels 5.3, 5.4 on the other side that carry fluid in the other direction which partition may be a flat heating element. The flat heating elements 54′, 54″ are preferably even, but may also be corrugated in flow direction alternatively. More than just two partial fluid channels may be provided in one direction also.

While in a working state the devices of this present invention advantageously permit a direct heat conductive connection to be achieved between a flat heating element and the fluid. It goes without saying that the advantages hereinbefore described may be availed of separately or in any desired combination. The exemplary embodiments hereinbefore described are not deemed to be final and complete, but are just to be regarded as examples for a better understanding of the invention.

A still further embodiment of a device according to this present invention for heating flowing fluids according to FIGS. 8 to 12d provides for a fluid housing 51 and a flexible flat heating element 52 as a heating unit which latter is arranged substantially between an upper half 51′ of the fluid housing 51 and a lower half 51″ thereof. Said housing halves 51′, 51″ are preferably made of a transparent plastic material and fixed to each other by ultrasonic welding or bonding. Upper housing half 51′ and lower housing half 51″ have one half-shell type muff 55 and/or 56 each on a first end 53 and an opposite second end 54 to form an inlet 57 and/or outlet 58 for the fluid after assembly. Inlet 57 and outlet 58 are hence formed to a first small side wall 59 (first side) of the fluid housing 51 and protrude sideways from said first end 53 and/or second end 54 of the fluid housing 51.

Upper housing half 51′ and lower housing half 51″ each have a plurality of elongate ribs 60 and/or 61 formed to an inner surface 62 and/or 63 of said housing half 51′ and/or 51″. Seven such ribs 60, 61 are provided in this exemplary embodiment which constitute a partition for the fluid channels 64, 64′, 64″ and hence determine the course of said fluid channels. The ribs 60, 61 are substantially linear and extend from the first end 53 to the second end 54 of the fluid housing 51.

Upper and lower fluid channels 64, 64′, 64″ are in alignment with one another with the flat heating element 52 separating the upper and lower fluid channels 64, 64′, 64″ in an intermediate plane between the upper housing half 51′ and the lower housing half 51″. Free face ends of opposing ribs 60 of the upper housing half 51′ and ribs 61 of the lower housing half 51″ confine the flat heating element 52 within a line section 65. The flat heating element 52 is floatingly arranged between the upper housing half 51′ and the lower housing half 51″ and by one face end preferably attached to the upper housing half 51′ and/or the lower housing half 51″ by mechanical means. The opposing ribs 60, 61 keep the flat heating element 52 in a preferably central intermediate position and prevent the flexible heating element 52 from swelling up under the action of the fluid flow.

The flat heating element 52 is in the form a flexible printed board having a heat conductor track section 66 and a marginal contact strip 67. Said contact strip 67 is arranged on a second narrow side wall 59′ (second side) of the flat heating element 52. The heat conductor track section 66 is disposed inside the fluid housing 51 whereas the contact strip 67 integral with the heat conductor track section 66 is arranged outside said housing 51 and serves to establish mechanical/electrical contact with a not-shown plug strip positioned in a slot of a control unit.

The flat heating element 52 has a number of electrically conductive heat conductor tracks each on a first flat side 68 and a second flat side 69. As will be seen in FIGS. 9 and 10 said first flat side 68 and said second flat side 69 has two heat conductor sections 71, 72 which are tandem arranged in a fluid main flow direction. The first heat conductor section 71 has a first heat conductor track 70 and sweeps substantially a first half (first heat conductor section 71) of the first flat side 68 and/or 69 with line sections 65 only exposed for engagement with the ribs 60, 61. The heat conductor track 70 of the first heat conductor section 71 leads to contacts K1, K2 of the contact strip while the heat conductor track 70′ of the second heat conductor section 72 leads to contacts K3, K4 of said contact strip 67.

Also provided are a connector 74 for another sensor in the area of the inlet 57 on the flat heating element 52, a connector 75 for a second temperature sensor in the area of the outlet 58, a connector 77 for a further sensor in the area of the face ends 76 of the flat heating element 52 and a connector 78 for another sensor in an area oriented toward the contact strip 67. Said further sensors may be provided as temperature sensors for assessing excess temperature conditions or such like. Bonding thereof to the connectors 74, 75, 76, 77, 78 is preferably by the way as usually done for temperature sensors. Another sensor may for instance be provided as fluid sensor which during initialization on startup of the flat heating element 52 detects whether fluid is present inside the fluid housing 51 or not.

The ribs 60, 61 are curved toward stub extensions 55 and/or 56 and form a deflecting section which at a first end 53 of the fluid housing 51 reverses the fluid entering in a center plane A of the fluid housing 51 into a main flow direction 73 and/or from the main flow direction 73 to an outlet 58 extending perpendicular to said center plane A. As will be seen, an inlet collecting area 79 is formed at the first end 53 of the fluid housing 51 from which the fluid channels 64, 64′, 64″ extend substantially linearly in main flow direction 73. On an opposite side, i.e. at a the second end 54, there is a fluid collecting area 80 provided in which the fluid coming from the fluid channels 64, 64′, 64″ is collected and conducted to the outlet 58 after a 90° inversion of the direction like at the first end 53 of the fluid housing 51. An axis of the inlet 57 and an axis of the outlet 58 hence extend substantially perpendicular to the fluid channels 64, 64′, 64″ and/or perpendicular to the main flow direction 73.

A width b, b′, b″ of fluid channels 64, 64′, 64″ is substantially constant in the main flow direction 73. Said width b, b′, b″ of fluid channels 64, 64′, 64″ however starts to reduce from the first narrow side wall 59 comprising the inlet 57 and the outlet 58 toward the contact strip 67. In this present exemplary embodiment, the width b of a first fluid channel 64 facing the first narrow side wall 59 is relatively large. Two adjacent fluid channels 64′ have a width b′ which is half that width. Further fluid channels 64″ extending up to the contact strip 67 in a juxtaposed relation have a further reduced width b″. This reduction of fluid channel width and/or fluid channel cross-sectional area ensures that the flat heating element 52 heats the fluid over identical lengths of time across the whole cross-sectional area of the fluid housing 51.

As may be best seen from FIG. 12d, the heat conductor tracks 70 of the first flat side 68 on the one hand and the heat conductor tracks 70′ of the second flat side 69 of the flat heating element 52 on the other hand are relatively offset in a transversal direction Q thereof such that as viewed in projection to an extension plane E of said heating element 52 they are disposed side by side. This makes sure that there is no temperature block in the flat heating element 52 and/or heat introduction is improved and more homogeneous. The heat conductor tracks in this present exemplary embodiment extend substantially perpendicular to the main flow direction 73 such that the transversal direction Q is substantially parallel to said main flow direction 73.

In this present exemplary embodiment there are two heat conductor track sections and/or two heating circuits arranged on each flat side 68, 69 of the flat heating element 52 which may be connected to a temperature control means via different contacts K. This temperature control means is arranged in an external control unit. The heating circuits 66 may be series or parallel connected which reduces the extent of control equipment.

A not-sown alternative embodiment provides for a rigid flat heating element with a rigid printed board to be used in lieu of a flexible flat heating element 52. The substrate material may be a phenolic or epoxy resin which is reinforced with paper and/or glass fabric if so deemed necessary.

The method for producing a flat heating element 52 will hereafter be described in closer detail: A polyimide film (for instance Kapton) having a thickness between 12 μm and 125 μm is used as a flexible substrate. A metallic foil 82 (copper foil) is applied to the two flat sides of the substrate 81, preferably by bonding and/or laminating, to provide an electrically conductive layer. Applying the metallic foil 82 to the substrate 81 is from a roll R1 to a roll R2 with a semi-finished material on a Roll R1, R2 being made available after the metallic foil 82 has been placed on the substrate 81. Alternatively, the metallic layer may be applied to the plastic substrate film by rolling or galvanizing or spraying.

In a next processing step the heat conductor tracks 70, 70′ are structured by photolithography for which purpose a photosensitive layer 83 (photosensitive resist) is applied to the metallic layer. The application process is continuous during payoff of the semi-finished material from roll R1 and rewinding there of on roll R2.

A further step comprises an exposure of the photosensitive layer 83 by means of a light source L via a mask M1, M2 which determines the subsequent course of the heat conductor tracks 70, 70′. Exposure under said mask M1, M2 hence provides masking of the photosensitive layer 83. A still further step is the development of the photosensitive layer 83 as per FIG. 12b with said photosensitive layer 83 to subsist only in those areas in which the heat conductor tracks 70, 70′ are to be located.

In a still further processing step may the metallic layer 82 be removed from the areas between the remainder of the photosensitive resist by etching—see FIG. 12c. A next step comprises to remove the photosensitive layer 83, in particular by rinsing, cleaning and subsequent drying—see FIG. 12d.

Structuring the heat conductor tracks 70, 70′ may be effected by lasing also in a not-shown alternative embodiment.

A solder paste may be applied to the connections 74, 75, 76, 77, 78 in a still further processing step to prepare for fitting the sensor elements by subsequent soldering (such as reflow or infrared radiation soldering).

The afore described working steps may be performed in a roll-to-roll process in which the semi-finished material is unwound from one roll R1 to the other roll R2 or vice-versa. Roll movement is momentarily stopped to allow time for the working step to be performed.

Another step relates to cutting off semi-finished material blanks 64 from the roll web which each constitute one flat heating element 52. This may for instance be done with the aid of a laser. The sized and outfitted semi-finished material blanks can then be provided with an additional protective layer of a biocompatible material and/or of an electrically insulating material and/or of a mechanical protective material and/or with a thermally conductive layer in a further processing step.

The flat heating element 52 can be inserted between and bonded to the housing halves 51′ and 51″ to form a heating unit 2.

A not-shown embodiment of the invention provides for the heat conductor tracks 70′, 70″ of the flat sides 68, 69 to be arranged in an overlapping relation rather than side by side as viewed in projection on the extension plane E of the substrate 81.

Fluids used may be gases or liquids such as blood, plasma or infusions as medical and/or human or animal liquids, or alternatively also cell cultures employed in laboratory technology, or even water or other liquids of the type used in the food processing industry.

It is possible in another alternative embodiment of this present invention to provide the flat heating element 52 with heat conductor tracks on just one flat side 69. The flat side without heat conductor tracks is preferably in a flat contact with an inner surface of one half of the fluid housing 51 in that case.

Another alternative embodiment of the invention provides for the substrate 81 (substrate film) to be completely provided with a thermally conductive layer such that temperature distribution throughout the area of the flat heating element 52 is very homogeneous.

Due to the fact that the flat heating element 52 is preferably provided with a fluid sensor it is possible during an initialization run on startup of the heating unit and/or the flat heating element 52 or at any other suitable moment to detect if there is a fluid and/or a liquid present in the system or not. This permits for instance a plausibility check to be made which indicates that the heating unit is ready for operation. Production tolerances of resistor tracks or such like also may be determined during initialization. A fluid sensor used may be a capacitor, for instance, whose capacity varies in response to changes of the dielectric constant as a function of moisture. Said capacitor may preferably be formed by etching the metallic layer 82 the way as done for the heat conductor tracks. In another embodiment it is possible to use the fluid sensor not only to detect fill levels, but also flow conditions or the presence of gas bubbles.

Other than the embodiment according to FIGS. 8 to 11 a further exemplary embodiment of the invention according to FIG. 13 provides not for a plate-shaped or flat fluid housing to be arranged, but for a fluid housing 91 of cylindrical shape in which a flat heating element 92 is disposed in a helically wound configuration. The flat heating element 92 of rectangular shape is helically arranged cross to a fluid direction 93 to thereby form annular fluid channels 94 which are parallel in said fluid direction 93. There is a fluid inlet 95 on an upstream face end 95 of the fluid housing 91 and a fluid outlet 96 on a downstream face end 96 thereof. A contact strip 97 protrudes from the interior of the fluid housing 91 in the region of a housing longitudinal side wall for bonding a heat conductor track applied to the flat heating element 92 to connections that leads to a control unit.

In another embodiment of the invention according to FIG. 14 a fluid housing 101 of rectangular and/or square cross-section may be provided in which a flat heating element 102 is wound in a substantially angular configuration to form parallel fluid channels 103 which extend substantially linear from an upstream face end of the fluid housing 101 to a downstream face end thereof as well. There is also an inlet 104 in the region of an upstream face end of the fluid housing 101 and an outlet 105 in the area of a downstream face end of the fluid housing. Same as described for the preceding exemplary embodiment there is a contact strip 106 arranged on a narrow side of the fluid housing 101. Other than in the previous exemplary embodiment are the fluid channels 103 not provided in an annular, but a rectangular and/or square configuration.

Alternatively, the substrate 81 may be a rigid substrate also.

In another not-shown embodiment of the invention can at least one sensor for detection of such parameters of a patient's body fluids as oxygen content and/or oxygenation in the blood of a patient be provided on a flexible or rigid printed board in lieu of or in addition to the temperature sensors 21, 22 and/or 23 The device has a multiple function in one embodiment, namely to make a fluid available that has a defined temperature due to the temperature sensor system on one hand while therapeutic processes may be supervised such as a blood purification treatment (dialysis) in case of failure of a patient's kidneys or other organs.

A further embodiment of the invention according to FIGS. 15 to 18 provides for a fluid housing 111 in which a flat heating element 112 with meander type heat conductor tracks 113, 113′ is arranged. Said flat heating element 112 is integral with a contact strip 114 which is disposed on a longitudinal side 115 of the fluid housing 111 on the outside thereof.

The fluid housing 111 consists of two fluid housing halves each comprising a plurality of spacer means 116 on an inner surface thereof. Said spacer means 116 are in the form of webs protruding from and distributed over said inner surface in such a way that the flat heating element 112 is disposed in an opening plane of the housing halves in a substantially even position. A flat fluid channel extends on both flat sides of the flat heating element 112, namely a first flat fluid channel 119′ between a first inner surface 117′ of the fluid housing 111 and/or the first fluid housing half 111′ and a first flat side 119′ of the flat heating element 112 on the one hand and a second flat fluid channel 119″ between a second inner surface 117″ of the fluid housing 111 and/or 111″ and a second flat side of the flat heating element 112 on the other hand. This means that there are two flat fluid channels 119′, 119″ provided in a symmetrical relation to a longitudinal center plane of the fluid housing 111 which have a relatively large flow space and a low flow resistance. The fluid entering through an inlet 121 at first face end 120 of the fluid housing 111 flows into the first and the second flat fluid channels 119′, 119″ in which it is directed to an opposite second face end 122 with substantially no directional change involved and essentially in longitudinal extension of the fluid housing 1 and/or in a linear stream is conducted to an opposite second face end 122 and finally to a transfer conduit again via the outlet 123. The fluid is carried along a flow direction F which is parallel to the longitudinal extension of the fluid housing 111.

The spacer means 116 may be dots or elongate webs in a not-shown alternative embodiment.

The fluid channels 119, 119′ extend linearly between the inlet 121 and the outlet 123 of the fluid housing 111, so to say in a straight extension of the fluid entry direction at the inlet 121. Each of the fluid channels 119′, 119″ has a width corresponding to that of the fluid housing 111. The fluid channels 119′, 119″ conduct the fluid between the inlet 121 and the outlet 123 of the fluid housing 111 in substantially parallel and linear streams with an inversion effected only at the inlet 121 and the outlet 123 due to different transversal extensions in those areas. The length of the fluid housing 111 between the inlet 121 and the outlet 123 is preferably greater than a width that corresponds to the distance between the two opposing longitudinal sides 115. The fluid channels 119, 119′ extend linearly between the inlet 121 and the outlet 123 of the fluid housing 111.

The fluid channels 119, 119′ are in mirror-symmetrical arrangement relative to the longitudinal center plane of the fluid housing 111 which plane intersects the inlet 121 and the outlet 123 of the fluid housing 111.

The flat heating element 112 has meander type heat conductor tracks 113, 113′ on both of its flat sides 118′, 118″ of which an example is represented for one flat side in FIG. 116. The heat conductor tracks 113, 113′ preferably extend in the flow direction F and/or parallel to the longitudinal edges 115 of the fluid housing on both sides of a transversal center plane Q of the flat heating element 112 and ends of said heat conductor tracks 113, 113′ facing the contact strip 114 are joined with respective contacts 124 of the contact strip 114 via contactor tracks 125 that extend linearly in the region of the transversal center plane Q. The risk of undesirable adherence of gas bubbles is eliminated due to the fact that said heat conductor tracks 113 extend in the flow direction F. Moreover, mechanical fatigue strength is being increased.

While the heat conductor tracks 113, 113′ each extend on both flat sides 118′ 118″ of the flat heating element 112, there is just one temperature sensor 126 arranged on a first face end 120, a second temperature sensor 127 on the opposite face end 122 and a third temperature sensor 128 to accesses an excess temperature condition on the first flat side 118′ in an area close to the transversal center plane Q. Alternatively, said third temperature sensor 128 for detecting an excess temperature condition may be arranged marginally in the area of the second face end 122, i.e. preferably in a place where the highest fluid temperature is expected to develop. Additional temperature sensors may be fitted if deemed necessary which should be arranged as close as possible to the conductor track for the sake of a quick temperature detection.

The fluid housing halves 111′ and 111″ are for instance joined by bonding, ultrasonic welding or spraying. Marginal edges of the housing halves 111′, 111″ are directly attached to the component consisting of flat heating element 112 and contact strip 114 via an adhesive sealant. The third temperature sensor 128 for detecting excess temperature conditions should preferably be disposed in the area of highest expected fluid temperature.

As will be seen from FIG. 18, a cassette 129 comprising the fluid housing 111, the flat heating element 113 and the contact strip 114 may be arranged in a preferably vertical slot 130 of a control unit 131. This control unit 131 has a casing 140 in which a control means 141 to control and/or regulate fluid heat-up, a monitor 142 and a power supply unit 143 (power pack) are arranged. Said receiving slot 130 is preferably provided with spring contact elements into which said contact strip 114 snaps by its contacts when in an operative position. The cassette 129 is hence mechanically and electrically connected to said control means 141 via this spring clip connection. While the contact strip 114 is concealed in said receiving slot 120 of the housing 140, the fluid housing 111 with flat heating element 112 is disposed externally of said casing 140 and visible from the outside such that variations, if any, in fluid consistency or flow properties may be recognized.

The heat conductor tracks 113 on the first flat side 118′ of the flat heating element 112 are offset relative to the heat conductor tracks 113 on the second flat side 118″ of said heating element 112 in the extension plane thereof and/or in the fluid flow direction F such that they are not overlapping each other and ensure a homogenous heat input into both fluid channels 119′, 119″.

Since several heat conductor tracks 113, 113′ on each flat side 118′, 118″ can be separately activated in the flow direction F with the aid of an electric control unit connected via a contact strip 114, different heat conductor sections are formed in said flow direction F which can be activated and/or deactivated in an alternating order and/or periodically. For example, a section disposed upstream in flow direction F may be activated while a downstream heating section is deactivated and vice-versa. This improves the input heat dosage.

Sensors for detecting a fill level or flow rate also may be arranged inside the fluid housing 111.

The flat heating element 112 is preferably flexible and consisting of a substrate on which heat conductor tracks 113, 113′ are formed by such methods as etching, printing or the like. In addition, the flat heating element 112 is coated with a biocompatible material.

The printed board of the flat heating element is always provided with an electrically insulating layer which is preferably biocompatible.

In a not-shown alternative embodiment of the invention the flat heating element 112 may be attached to an inner surface of the fluid housing ill also in which case just one fluid channel extends inside said fluid housing 111.

In another alternative embodiment not shown can the flexible printed board be a so-called “rigid-flexible” board connected to a rigid contact strip.

The features hereinbefore described of the exemplary embodiments may be used separately or jointly in any desired combination according to a further embodiment. They are not to be deemed to be final and complete, but are just to be regarded as examples for a better understanding of the invention.

1.-27. (canceled)

28. A device for heating flowing fluids, in particular intravenous fluids, comprising a fluid housing containing at least one fluid channel through which the fluid can be conducted from an inlet of the fluid housing to an outlet of the fluid housing, a heating unit comprising at least one electric flat heating element for heating fluid that flows through the fluid channel and a temperature control unit containing at least one temperature sensor arranged on the flat heating element, characterized in that the flat heating element comprises a flexible or rigid printed board with heat conductor tracks arranged on the first flat side (118′) and/or second flat side (118″) wherein the flat heating element is arranged inside the fluid housing and at least partially forms a wall of the fluid channel, the fluid housing is profiled such as to form a meander or spiral type fluid channel.

29. The device according to claim 28, wherein the flat heating element (112) is integral with a contact strip (114) that extends outside the fluid housing (111) along a longitudinal side (115) thereof.

30. The device according to claim 28, wherein heat conductor tracks (113, 113′) arranged on the first flat side (118′) and/or the second flat side (118″) of the flat heating element (112) are meandering from the contact strip (114) to an opposite longitudinal side of the fluid housing (111).

31. The device according to claim 28, wherein the fluid housing (111) consists of two housing halves (111, 111″) whose marginal edges are jointed to each other and in the region of the contact strip (114) attached to said latter by bonding or ultrasonic welding or spraying.

32. The device according to claim 28, wherein the flat heating element (52) is disposed between and spaced from a first inner surface (62) and a second inner surface (63) of the fluid housing (51) in a substantially floating arrangement.

33. The device according to claim 28, wherein on a first flat side (68) and a second flat side (69) each at least one hat conductor track (70, 70′) is arranged wherein the heat conductor tracks (70, 70′) of said first flat side (68) and said second flat side (69) are parallel to each other.

34. The device according to claim 28, wherein on the flexible or rigid printed board (19, 81) there is a first temperature sensor (21) arranged in the region of the inlet of the fluid channel (5) and/or a second temperature sensor (22) in the region of the outlet (11) of the fluid channel (5) and/or a third temperature sensor (23) for assessing an excess temperature condition and/or a fluid sensor for detecting fluid presence and/or a sensor for assessing a fluid flow rate.

35. The device according to claim 28, wherein a quotient of effective heat transfer area of the flat heating element (4) divided by an area of the flat heating element (4) projected on a longitudinal center plane of the fluid housing (1) is larger than 1.

36. The device according to claim 28, wherein the fluid housing (111), the flat heating element (112) and the contact strip (114) are combined into a single-use cassette (129).

37. The device according to claim 36, wherein the cassette (129) can be inserted into a receiving slot (130) of a control unit (131) wherein while in such inserted position the contact strip (114) is electrically and/or mechanically snap connected to mating contact elements and wherein the fluid housing (111) is at least partially arranged outside the receiving slot (130).

38. The device according to claim 28, wherein the receiving slot (130) of the control unit extends vertically such that the cassette (129) is in upright position while inserted and the fluid flow direction is perpendicular.

39. A method for producing a flat heating element used for heating flowing fluids, in particular as disclosed in any of the preceding claims 1 to 11, characterized by the following steps: applying an electrically conductive layer (82) to one and/or two flat sides of a substrate (81) to provide a semi-finished material made available from a roll; sequential structuring of heat conductor tracks (80, 70′) on sections (84) of the semi-finished material web by lasing or photolithographic means while a section (84) of the semi-finished material web is being paid off from one roll (R1) and wound up again on another roll (R2); cutting off a blank from the semi-finished material web (84) which has the size of a flat heating element (52) while the web is being paid off from the roll (R1, R2); and covering the flat heating elements (52) with a biocompatible material and/or and an electrically insulating material and/or a mechanical protective material and/or a thermally conductive coating.

40. The method according to claim 39, wherein the semi-finished material made available from a roll is being paid off and wound up for several processing steps to be performed, namely a first step in which a photosensitive layer (83) is applied to the semi-finished material, a second step in which the photosensitive layer (83) is masked, a third step in which said photosensitive layer (83) is developed, a fourth step in which the electrically conductive layer (82) is removed from areas not covered by the photosensitive layer (83) to form the heat conductor tracks (70, 70′) and a fifth step in which the photosensitive layer (83) is removed from the heat conductor tracks (70, 70′).

41. The method according to claim 39, wherein the heat conductor tracks (70, 70′) on both flat sides (68, 69) of the semi-finished material are structured with such relative offsets that in projection on the substrate (81) they are overlapping each other or arranged side by side.

42. The method according to claim 40, wherein the flat heating element (52) is fitted with a fluid sensor to detect the presence or absence of fluid in the system.
